(19) United States
(12) Patent Application Publication
(10) Pub. No.: US 2023/0158046 A1
(43) Pub. Date: May 25, 2023

(54) PHYTOECDYSONES FOR USE IN THE PREVENTION OF MUSCLE STRENGTH LOSS DURING IMMOBILISATION

(71) Applicants: BIOPHYTIS, Paris (FR); SORBONNE UNIVERSITE, Paris (FR)

(72) Inventors: Pierre DILDA, Paris (FR); Mathilde LATIL, Charenton Le Pont (FR); René LAFONT, Paris (FR); Stanislas VEILLET, Savigny Sur Orge (FR)

(21) Appl. No.: 18/158,859

(22) Filed: Jan. 24, 2023

Related U.S. Application Data
(62) Division of application No. 16/976,189, filed on Aug. 27, 2020.

(30) Foreign Application Priority Data
Feb. 28, 2018 (FR) 1851778

(51) Int. Cl. A61K 31/575; A61P 21/06; A61K 9/00; A61K 31/57

(52) U.S. Cl. CPC A61K 31/575; A61P 21/06; A61K 9/0053; A61K 31/57

(57) ABSTRACT

A composition including at least one phytoecdysone or at least one semi-synthetic derivative of a phytoecdysone, for use in mammals for preventing the loss of muscle strength during immobilisation. More particularly, a composition for use that includes 20-hydroxyecdysone or a semi-synthetic derivative of 20-hydroxyecdysone. Moreover, the composition for use includes a compound of general formula (I).

### CROSS REFERENCE TO RELATED APPLICATIONS

This application is a division of Application No. 16/976,189, filed Aug. 27, 2020, which is a National Stage of International Application No. PCT/FR2019/050392, having an International Filing Date of 20 Feb. 2019, which designated the United States of America, and which International Application was published under PCT Article 21(2) as WO Publication No. 2019/166717 A1, which claims priority from and the benefit of French Patent Application No. 1851778, filed on 28 Feb. 2018, the disclosures of which are incorporated herein by reference in their entireties.

### BACKGROUND

### 1. FIELD

The present disclosure relates to the use of phytoecdysones and derivatives thereof for use in the prevention of muscle strength loss during immobilisation.

### 2. BRIEF DESCRIPTION OF RELATED DEVELOPMENTS

The risk of complications following the immobilisation of a limb or the maintenance of a lying position over the long term (decubitus) is high and may be manifested by muscle disorders, but also bronchopulmonary, cardiovascular or osteoarticular problems (Bodine 2013, Hermans & Van den Berghe 2015; De Jonghe et al. 2007; Wentworth et al. 2017; lordens et al. 2017).

The immobilisation may be gradual or abrupt and may relate to a limb or extend to the entire body in the most extreme cases. The circumstances resulting in immobilisation are many, for example:
fracture or injury of a limb leading to the fitting of an external immobilisation device (non-plastered supports of the cervical collar or sling type, an immobilisation bandage, metal or knee braces, syndactyly or plaster supports with complete or incomplete immobilisation of a limb),
partial or total ligament rupture requiring or not surgical intervention leading to the fitting of an external immobilisation device,
fitting of a hip prosthesis,
fitting of a knee prosthesis,
orthopaedic fittings not allowing immediate bearing,
fracture of the pelvis in a painful period,
fracture of the neck of the femur, operated or unoperated, stable or unstable,
partial peripheral nervous attack due to a trauma,
partial attack of the spinal cord due to a trauma, and
patients placed in a decubitus position for a prolonged period.

In all cases, immobilisation will lead to alterations to the muscle tissue. A reduction in the muscle mass is observed, along with muscle wasting or amyotrophy, as well as a reduction in muscle strength and power, which generally leads to a period of incapacity and in certain cases to a prolongation of care causing an increase in hospital costs.

Atrophy and loss of skeletal muscle strength following immobilisation have functional consequences in particular on posture and balance, which increases the risk of falling, in particular in aged persons (Onambele et al., 2006).

In France, it is estimated that per year there are between 50,000 and 80,000 fractures of the upper end of the femur in old people. The very great majority of these fractures of the femur are consecutive upon falls (INSERM 2015). Fractures of the neck of the femur lead to a transient loss of mobility, which may cause the occurrence of complications. The fracture of the end of the femur is one of the main causes of mortality in those over 65 years of age (Gillespie et al. 2012). In the year following the accident, mortality is 10% to 20% higher than that in subjects of the same age and the same sex. All causes taken together, from 20% to 30% of patients aged over 55 years die in the year following a fracture of the upper end of the femur (Klop et al., 2014 and Lund et al., 2014).

In an aged subject muscular atrophy following immobilisation has very serious consequences, which may be aggravated by muscular atrophy in combination with aging (sarcopenia). The mechanisms involved in muscular atrophy related to aging and in muscular atrophy related to immobilisation are however different (reviewed in Lynch et al., 2007; Romanick & Brown-Borg, 2013).

During immobilisation, it can be observed that it is the type I (oxidative) fibres that are particularly affected by the atrophy (Bigard et al., 1998; Ohira et al., 2006). On the other hand, sarcopenia manifests in a particular atrophy concerning type II (glycolytic) fibres, associated with the development of conjunctive tissue (fibrosis) and an infiltration by adipose cells (e.g. Nilwik et al., 2013). Sarcopenia is characterised by a reduction in the diameter of the fibres and the number thereof (Lexell, 1993). On the other hand, in a context of atrophy related to immobilisation, the size of the fibres decreases but the number of muscle fibres remains constant (Narici, 2010). In addition, atrophy involves processes of autophagia, the regulation of which differs according to the type of fibre (Yamada et al., 2012).

The molecular mechanisms that underlie the muscular atrophy caused are also different. Some genes known to cause muscular atrophy, such as MuRF1 and Atrogin, can be activated by well-known signalling pathways such as NF-κB. This signalling pathway is activated under atrophy conditions related to cachexia or to immobilisation (Hunter et al., 2002) but not in a context of sarcopenia (Bar-Shai et al., 2005; Phillips, 2005; Sakuma, 2012).

Myostatin, a negative regulator of muscle growth, increases during atrophy related to cachexia and during immobilisation but it has been demonstrated that this was not the case in aged animals, in which the myostatin level remained relatively unchanged (Siriett et al., 2006; Lebrasseur et al., 2009).

Under these conditions, the fact that a substance is effective for treating sarcopenia does not make it possible to predict that it will be effective for preventing muscle disorders related to immobilisation.

Immobilisation also has an impact on the recovery of maximum physical performance, in particular in athletes (Milsom et al. 2014).

The plastered immobilisation of an injured limb causes structural modifications of the muscles involved in the immobilisation. For example, two months of immobilisation of the ankle lead to a reduction in the volume of the triceps surae and of the quadriceps respectively of 21.9% and 24.1%. Two months after the end of the immobilisation, the two muscles are still 9.5% and 5.2% less voluminous than before immobilisation (Grosset & Onambele-Pearson, 2008). In terms of cross-sectional area (CSA) of muscle fibres, five weeks of immobilisation reduces the CSA by 10% to 20% depending on the type of fibre and the muscles concerned (Suetta et al. 2004; Berg et al. 2007). The CSA of muscles supporting body weight decreases approximately by 2-3% per week during the first months of immobilisation (Berg et al. 2007).

Muscle strength is also greatly reduced following immobilisation. In general, an immobilisation of the leg for two weeks produces a loss of one third of the muscle strength in young people, whilst older subjects lose approximately one quarter of their strength. In the latter, the loss of muscle power may prove to be irreversible. This may give rise to a loss of confidence and weakness invariably leading to dependency. In addition, the appearance of an immobilisation syndrome may follow confinement to bed or simply a great reduction in activity.

Patients confined to bed for more than a week have a loss of muscle strength of their anti-gravitational muscles of the calves and back (Hermans et Van den Berghe, 2015). In a healthy person, during the first week of confinement to bed, losses of strength of between 1% to 6% per day have been observed (Appell, 1990). A period of 1 to 2 weeks of inactivity of the lower limbs causes a reduction in 15% of the strength of the extensors of the knee in subjects aged around twenty.

In addition, a study demonstrated that fitting plaster for 5 days caused a loss of strength in the quadriceps of 9% while this loss reached 23% after 14 days of immobilisation. Confinement to bed for 3 months has very marked effects on the force developed with a reduction of 31% to 60% according to the muscle in question (Alkner & Tesch, 2004).

The revelation of the deleterious role of muscle immobilisation has led to testing the feasibility and efficacy of several preventive methods. Thus, in some cases, in order to overcome the muscle damage caused by immobilisation, early active or passive remobilisation programmes for patients have been established. These programmes have the drawback of being difficult to introduce on a large scale. In addition, use of these methods is tricky or even impossible in some surgical patients with whom mobilisation proves to be painful.

Studies of muscle electrostimulation have also been carried out, but this approach does not make it possible to have a systemic beneficial effect of stimulation, it may prove ineffective in patients presenting with inexcitability of the muscle membrane and the choice of the muscle territory to be stimulated is tricky in cases of extended immobilisation.

Apart from the tissular and mechanical changes to the muscle during immobilisation, metabolic and major molecular changes also arise.

Thus protein synthesis is reduced, while immobilisation causes an increase in oxidative stress, inflammation, apoptosis and activation of proteolytic pathways that lead to degradation of muscle proteins.

Phytoecdysones represent an important family of polyhydroxylated sterols. These molecules are produced by various species of plants and participate in the defence of these plants against pests. The main phytoecdysone in the plant kingdom is 20-hydroxyecdysone. Studies have highlighted the anti-diabetic and anabolic properties of some phytoecdysones. Stimulating effects on protein syntheses in the muscles are observed in rats in vivo (Syrov, 2000; Toth et al., 2008; Lawrence, 2012) and on mouse C2C12 myotubes in vitro (Gorelick-Feldman et al., 2008).

Semisynthetic derivatives of 20-hydroxyecdysone were proposed in the publication of the French patent application of the applicant company, published under the number FR 3021318 A1.

### SUMMARY

The objective of the present disclosure is to limit as far as possible the loss of muscle strength during immobilisation, in particular following for example a fracture, confinement to bed or simply a great reduction in activity.

The inventors have discovered that phytoecdysones and derivatives thereof protect against the loss of muscle strength related to immobilisation. Muscle strength is defined by the absolute and specific maximum isometric force of the skeletal muscle.

Unexpectedly, phytoecdysones and derivatives thereof significantly reduce the loss of muscle strength related to immobilisation without this property being related to an anabolising effect on the skeletal muscle.

The present disclosure relates to phytoecdysones and derivatives thereof intended to be used in the treatment of disorders resulting from an alteration in the muscle function caused by immobilisation.

In the remainder of the description, phytoecdysones and derivatives thereof mean extracts of plants rich in 20-hydroxyecdysone, and compositions including 20-hydroxyecdysone by way of active agent.

Said plant extracts rich in 20-hydroxyecdysone are for example extracts of Stemmacantha carthamoides or Cyanotis vaga.

The extracts obtained are preferably purified to pharmaceutical grade.

The disclosure relates to a composition including an ecdysteroid, for use thereof in mammals for preventing loss of muscle strength during immobilisation.

More precisely, the disclosure relates to a composition including at least one phytoecdysone and at least one semisynthetic derivative of a phytoecdysone, for use thereof in mammals for preventing loss of muscle strength during immobilisation.

In aspects of the disclosure the composition includes 20-hydroxyecdysone or a semisynthetic derivative of 20-hydroxyecdysone. One example of such a composition is the extract, purified to pharmaceutical grade, BIO101 that has been developed by the applicant. BIO101 is a plant extract, said plant being chosen from plants containing at least 0.5% 20-hydroxyecdysone by dry weight of said plant, said extract including at least 95%, and preferably at least 97%, 20-hydroxyecdysone.

In aspects of the disclosure the composition includes a compound of general formula (I):

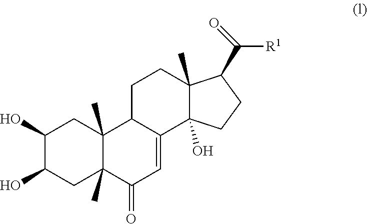

wherein:

R¹ is chosen from: a (C₁-C₆)W(C₁-C₆) group; a (C₁-C₆)W(C₁-C₆)W(C₁-C₆) group; a (C₁-C₆)W(C₁-C₆)CO₂(C₁-C₆) group; a (C₁-C₆)A group, A representing a heterocycle, optionally substituted by a group chosen from OH, OMe, (C₁-C₆), N(C₁-C₆), CO₂(C₁-C₆); a CH₂Br group;

W being a heteroatom chosen from N, O and S, preferably O and even more preferentially S.

In aspects of the disclosure the composition includes a compound chosen from the following compounds:

n° 1: (2S,3R,5R,10R,13R,14S,17S)-2,3,14-trihydroxy-10,13-dimethyl-17- (2-morpholinoacetyl)-2,3,4,5,9,11,12,15,16,17-decahydro-1H-cyclopenta[a]phenanthren-6-one, n° 2: (2S,3R,5R,10R,13R,14S,17S)-2,3,14-trihydroxy-17-[2-(3-hydroxypyrrolidin-1-yl)acetyl]-10,13- dimethyl-2,3,4,5,9,11,12,15,16,17-decahydro-1H-cyclopenta[a]phenanthren-6-one;

n° 3: (2S,3R,5R,10R,13R,14S,17S)-2,3,14-trihydroxy-17-[2-(4-hydroxy-1- piperidypacetyl]-10,13-dimethyl-2,3,4,5,9,11,12,15,16,17-decahydro-1H-cyclopenta[a]phenanthren-6-one;

n° 4: (2S,3R,5R,10R,13R,14S,17S)-2,3,14-trihydroxy-17-[2-[4-(2-hydroxyethyl)-1-piperidyl]acetyl]-10,13-dimethyl-2,3,4,5,9,11,12,15,16,17-decahydro-1H-cyclopenta[a]phenanthren-6-one;

n° 5: (2S,3R,5R,10R,13R,14S,17S)-17-[2-(3-dimethylaminopropyl(methyl)amino)acetyl]-2,3,14-trihydroxy-10,13-dimethyl-2,3,4,5,9,11,12,15,16,17-decahydro-1H-cyclopenta[a]phenanthren-6-one;

n° 6: ethyl 2-[2-oxo-2-[(2S,3R,5R,10R,13R,14S,17S)-2,3,14-trihydroxy-10,13-dimethyl-6-oxo-2,3,4,5,9,11,12,15,16,17-decahydro-1H-cyclopenta[a]phenanthren-17-yl]ethyl]sulfanylacetate; n° 7: (2S,3R,5R,10R,13R,14S,17S)-17-(2-ethylsulfanylacetyl)-2,3,14-trihydroxy-10,13-dimethyl-2,3,4,5,9,11,12,15,16,17-decahydro-1H-cyclopenta[a]phenanthren-6-one;

n° 8: (2S,3R,5R,10R,13R,14S,17S)-2,3,14-trihydroxy-17-[2-(2-hydroxyethyl sulfanyl)acetyl]-10,13-dimethyl- 2,3,4,5,9,11,12,15,16,17-decahydro-1 H cyclopenta[a]phenanthren-6-one.

In aspects of the disclosure the composition includes a compound of formula (II):

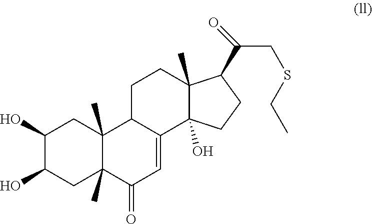 [SMILES: CCSCC(=O)[C@H]1CC[C@@]2(O)C3=CC(=O)[C@]4(C)C[C@@H](O)[C@@H](O)C[C@]4(C)C3CC[C@]12C]

In aspects of the disclosure, the composition is incorporated in a pharmaceutically acceptable formulation that can be administered orally.

In aspects of the disclosure, the phytoecdysones are administered at a dose of between 50 and 1000 milligrams per day in humans.

In aspects of the disclosure, the compound of formula (II) is administered at a dose of between 50 and 1000 milligrams per day in humans.

In aspects of the disclosure, the composition is administered during immobilisation. Preferentially, the composition is administered as from the first day of immobilisation.

In aspects of the disclosure, the composition is administered until immobilisation ends.

In aspects of the disclosure, the composition is further administered during a predetermined period after the end of immobilisation.

In an example aspect of the disclosure, the predetermined duration of treatment after the immobilisation ends corresponds to the time necessary for recovering a strength threshold corresponding for example to 80% or 100% of the estimated initial strength of the subject.

In an example aspect of the disclosure, the predetermined treatment period corresponds to a period of at least 28 days.

In an example aspect of the disclosure, the predetermined duration of treatment is a period of three to six months.

Preferentially, the treatment after the end of immobilisation is supplemented with a programme of physical exercises.

### BRIEF DESCRIPTION OF THE DRAWINGS

Other particular advantages, aims and features of the present disclosure will emerge from the following non-limitative description of at least one particular aspect of the object of the present disclosure, with regard to the accompanying drawings, wherein:

FIG. 1A is an image representing histological sections coloured with haematoxylin and eosin of anterior tibial (AT) muscle of a mouse of genetic background C57BL/6J non-immobilised,

FIG. 1B is an image representing histological sections coloured with haematoxylin and eosin of anterior tibial (AT) muscle of a mouse of genetic background C57BL/6J immobilised and treated with the vehicle for 14 days,

FIG. 1C is an image representing histological sections coloured with haematoxylin and eosin of anterior tibial (AT) muscle of a mouse of genetic background C57BL/6J immobilised and treated with the compound of formula (II) for 14 days.

FIG. 1D is a diagram representing the area of the muscle fibres of the anterior tibial muscle of a mouse of genetic background C57BL/6J non-immobilised (control), immobilised and treated with the vehicle for 14 days or immobilised and treated with a compound of formula (II) for 14 days,

FIG. 2A is a diagram representing the weight of the anterior tibial muscle of groups of mice of genetic background C57BL/6J non-immobilised (control), immobilised and treated with the vehicle for 14 days or immobilised and treated with the compound of formula (II) for 14 days.

FIG. 2B is a diagram representing the weight of the gastrocnemius muscle of groups of mice of genetic background C57BL/6J non-immobilised (control), immobilised and treated with the vehicle for 14 days or immobilised and treated with the compound of formula (II) for 14 days.

FIG. 3A depicts the absolute maximum isometric force of the anterior tibial muscle of a mouse of generic background C57BL/6J at various times post-immobilisation: non-immobilised (J0), after 14 days of immobilisation (J14), after 14 days of immobilisation and 1 week of remobilisation (J21) or after 14 days of immobilisation and 2 weeks of remobilisation (J28), treated with the vehicle or with the compound of formula (II).

FIG. 3B depicts the specific maximum isometric force of the anterior tibial muscle of a mouse of genetic background C57BL/6J at various times post-immobilisation: non-immobilised (J0), after 14 days of immobilisation (J14), after 14 days of immobilisation and 1 week of remobilisation (J21) or after 14 days of immobilisation and 2 weeks of remobilisation (J28), treated with the vehicle or with the compound of formula (II).

FIG. 4A is a diagram representing the weight of the anterior tibial muscle of groups of mice of genetic background C57BL/6J non-immobilised (control, measured at JO on non-immobilised animals), immobilised and treated with the vehicle for 14 days or immobilised and treated with the compound BIO101 for 14 days.

FIG. 4B is a diagram representing the weight of the gastrocnemius muscle of groups of mice of genetic background C57BL/6J non-immobilised (control), immobilised and treated with the vehicle for 14 days or immobilised and treated with the compound BIO101 for 14 days.

FIG. 5A is a diagram depicting the absolute maximum isometric force of the anterior tibial muscle of a mouse of genetic background C57BL/6J non-immobilised (control, measured at J0 on non-immobilised animals), immobilised and treated with the vehicle for 7 days or immobilised and treated with the compound BIO101 for 7 days.

FIG. 5B is a diagram depicting the specific maximum isometric force of the anterior tibial muscle of a mouse of genetic background C57BL/6J non-immobilised (control, measured at J0 on non-immobilised animals), immobilised and treated with the vehicle for 7 days or immobilised and treated with the compound BIO101 for 7 days.

FIG. 6A depicts the absolute maximum isometric force of the anterior tibial muscle of a mouse of genetic background C57BL/6J at various times post-immobilisation: non-immobilised (JO), after 7 days of immobilisation (J7), after 14 days of immobilisation (J14), and after 14 days of immobilisation and then 2 weeks of remobilisation (J28), treated with the vehicle or with the compound BIO101.

FIG. 6B depicts the specific maximum isometric force of the anterior tibial muscle of a mouse of genetic background C57BL/6J at various times post-immobilisation: non-immobilised (J0), after 7 days of immobilisation (J7), after 14 days of immobilisation (J14), and after 14 days of immobilisation and then 2 weeks of remobilisation (J28), treated with the vehicle or with the compound BIO101.

### DETAILED DESCRIPTION

### Method for Synthesising the Compound of Formula (II)

The compound of formula (II) to which reference is made in the rest of the description is as follows:

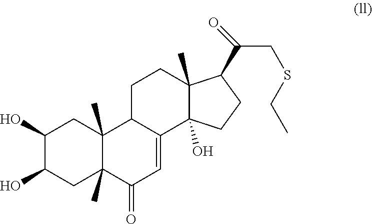 [SMILES: CCSCC(=O)[C@H]1CC[C@@]2(O)C3=CC(=O)[C@]4(C)C[C@@H](O)[C@@H](O)C[C@]4(C)C3CC[C@]12C]

The compound of formula (II) is obtained by semisynthesis from 20-hydroxyecdysone and then purification to pharmaceutical grade.

The method for preparing the compound of formula (II) by semisynthesis includes in particular:
a step of oxidising cutting of the side chain of the 20-hydroxyecdysone between carbons C20 and C22 in order to obtain poststerone,
a step of introducing a bromine atom at position C21, and
a step of reacting the bromine derivative with ethanethiol.

### Biological Activity of the Compound of Formula (II)

A model of immobilisation of a posterior paw of a mouse of genetic background C57BL/6J was implemented by means of a tube (Lang et al., 2012).

Female C57BL/6J mice aged 13 weeks were used. Ten mice were sacrificed at J0, these mice were not immobilised in order to serve as a control.

J0, J14, J21, J28 means the time elapsed as from the start of the experiment, expressed in days. Thus J0 designates the start of the experiment (before treatment and before immobilisation), J14 designates the 14th day as from the start of the experiment, etc.

Two groups of mice were formed, a test group and a reference group. Each group is exposed, orally, chronically either to the vehicle (reference group) or to the compound of formula (II) at a dose of 50 mg/kg per day (test group). The oral treatment over 28 days consists of tube feeding for five days per week and in drinking water for two days per week.

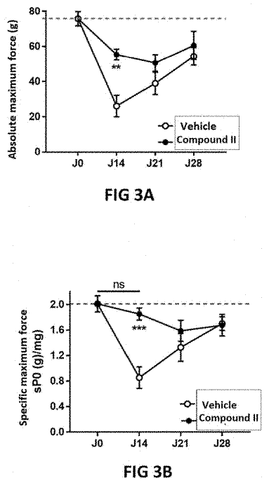

The animals in all the groups were tested for their functional capacity in situ by means of measurements of the absolute and specific maximum isometric force of the anterior tibial (AT) muscle (FIGS. 3A and 3B) after 14 days of immobilisation (n=13 for the vehicle, n=10 for the compound of formula (II)), after 14 days of immobilisation and one week of remobilisation (n=7 for the vehicle, n=8 for the compound of formula (II)) and after 14 days of immobilisation and two weeks of remobilisation (n=6 for the vehicle, n=8 for the compound of formula (II)).

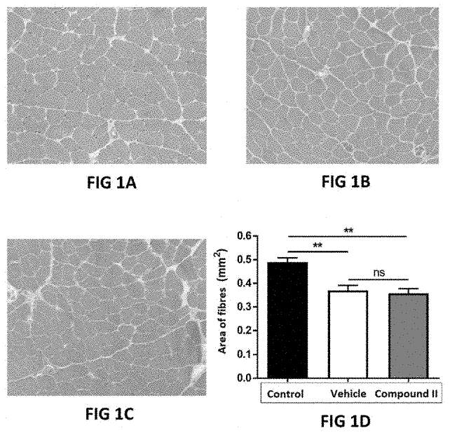

### Histology and Atrophy of the Muscles (FIG. 1)

A histological study of the anterior tibial muscle is carried out on sections coloured with haematoxylin and eosin (HE). The area of the muscle fibres is evaluated on control-mouse muscles, or treated with the vehicle or with the compound of formula (II). The muscle in all cases presents a histology of healthy muscle tissue (FIGS. 1A to C); on the other hand, as might be expected, after 14 days of immobilisation, the mean area of the fibres is greatly reduced in animals that received the vehicle compared with the control animals (−24.4%, p=0.006) that have not been immobilised. The area of the muscle fibres of the group treated with the compound of formula (II) is also reduced compared with the control group (−26.8%, p=0.002).

No significant difference is therefore observed between the groups of animals treated with the vehicle and the group treated with compound of formula (II) (p=0.73). After 14 days of immobilisation, the compound of formula (II) therefore does not exert any protective effect against loss of muscle volume.

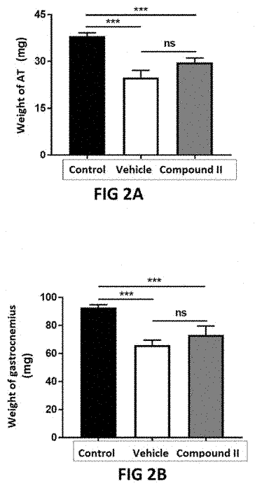

### Weight of the Anterior Tibial (AT) and Gastrocnemius Muscles (FIG. 2)

The weight of the AT muscles (FIG. 2A) and gastrocnemius muscles (FIG. 2B) were evaluated in non-immobilised (control) mice, and after 14 days of immobilisation in mice treated either with the vehicle or with the compound of formula (II), during the 14 days of immobilisation.

As expected, it is observed that immobilisation causes a reduction in the muscle mass of the AT and of the gastrocnemius in mice that received the vehicle compared with the control group (−34.9%, p<0.001 and −29%, p<0.001 respectively).

It is observed that the weight of the AT and gastrocnemius muscles does not vary significantly in the group of mice treated with the compound of formula (II) compared with the vehicle. Consistent with the results obtained on the diameter of the muscle fibres (FIG. 1), the compound of formula (II) therefore does not exert any protective effect against the loss of muscle mass following an immobilisation.

### Absolute and Specific Maximum Isometric Force of the Anterior Tibial Muscle (in situ Functional Study (FIG. 3))

An evaluation of the in situ contractility of the AT muscle is carried out at different times in the protocol: on non-immobilised control mice (J0), on mice subjected to an immobilisation of the posterior paw for 14 days (J14), immobilised for 14 days and then remobilised for 1 week (J21) and immobilised for 14 days and then remobilised for 2 weeks (J28).

On the day of sacrifice, the mouse is anaesthetised with an intraperitoneal injection of pentobarbital (55 mg/kg, 0.1 ml/10 g of body weight) before measuring the in situ force of the anterior tibial (AT) muscle. The skin on the top of the paw is incised, which reveals the tendon, which is cut at the distal end thereof. The distal tendon of the AT is attached to the lever of the servomotor (305B Dual-Mode Lever, Aurora Scientific). The skin on the lateral face of the thigh is incised, which reveals the sciatic nerve, between two muscle groups. The sciatic nerve is stimulated with a bipolar electrode (supramaximal pulse with a square wave of 10 V, 0.1 ms). The force is measured during contractions in response to the electrical stimulation (frequency of 75-150 Hz, duration 500 ms). The temperature of the mouse is maintained at 37° C. by means of a radiant lamp. The absolute maximum isometric force is measured (FIG. 3A) and the specific maximum isometric force (FIG. 3B) is calculated by comparing the absolute isometric force with the weight of the anterior tibial muscle.

As expected, it is found that the animals treated with the vehicle have an absolute maximum isometric contraction force significantly less than that of the non-immobilised control animals (−65.6%, p<0.001) (FIG. 3A). The animals treated with the compound of formula (II) exhibit a lesser absolute force loss (−26.9%, p=0.015) compared with the control, than the animals treated with the vehicle.

Surprisingly, it is observed that the treatment with the compound of formula (II) enables the animals immobilised for 14 days to preserve an absolute isometric force that is significantly greater than the animals treated with the vehicle and improves their performance (+112.1%, p=0.0041). This despite the absence of any effect of the compound of formula (II) on the loss of mass and muscle volume noted previously.

It is observed that the animals treated with the vehicle have a specific maximum isometric contraction force (sP0; FIG. 3B) significantly less than that of the non-immobilised control animals (−57.8%, p<0.001). Remarkably, the specific force of the animals treated with the compound of formula (II) is not significantly affected by the immobilisation: −8% (p=0.32) compared with the animals in the control group that were not immobilised. The treatment with the compound of formula (II) enables the animals immobilised for 14 days to preserve a normal muscle function while doubling the specific isometric force (+117.6%, p<0.001) compared with the animals in the immobilised group, treated with the vehicle.

### Biological Activity of the Compound BIO101

A second study was carried out using the same method of immobilisation of the posterior paws, on mice with the same age (13 weeks) and the same genetic background (C57BL/6J) as described previously, but adding an analysis point after 7 days of immobilisation. The analysis points are therefore J0, J7, J14 and J28.

Ten mice were sacrificed at J0, these mice were not immobilised in order to serve as controls (control group in the Figures).

J7, J14, J28 means the time elapsed as from the start of the experiment, expressed in days. Thus J7 designates the 7^{th} day as from the start of the experiment, etc.

Two groups of mice were formed, a test group and a reference group. Each group is exposed, orally, chronically either to the vehicle (reference group) or to the compound BIO101 at a dose of 50 mg/kg per day (test group). Compound BIO101 means a plant extract, said plant being chosen from plants containing at least 0.5% 20-hydroxyecdysone by dry weight of said plant, said extract including by way of active agent 20-hydroxyecdysone in a quantity of at least 95%, and preferably at least 97% by weight with respect to the total weight of the extract. The oral treatment for 28 days consists of tube feeding for five days per week and administration in drinking water for two days per week.

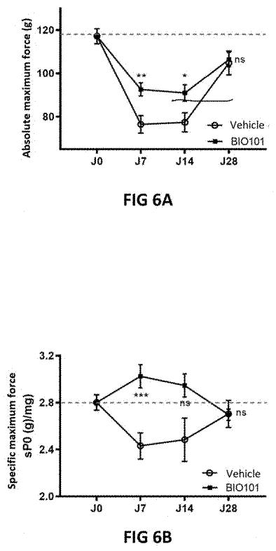

The animals in all the groups were tested for their functional capacity in situ (the two posterior paws) by means of measurements of the absolute and specific maximum isometric force of the anterior tibial (AT) muscle (FIGS. 6A and 6B) after 7 days of immobilisation (n=6 mice, two values per mouse for the reference group (vehicle), n=6 mice, two values per mouse for the test group (BIO101), after 14 days of immobilisation followed by two weeks of remobilisation (n=6 per mouse, two values per mouse for the vehicle (reference group), n=6 mice, two values per mouse for the compound BIO101), and after 14 days of immobilisation followed by two weeks of remobilisation (n=6 per mouse, two values per mouse for the vehicle (reference group), n=6 mice, two values per mouse for the compound BIO101).

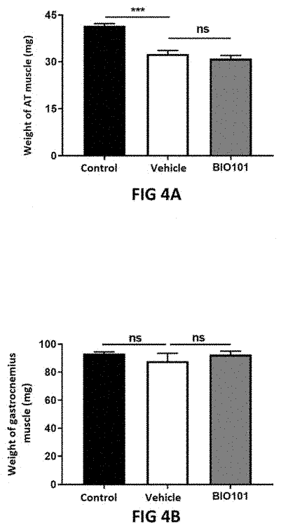

### Weight of the Anterior Tibial (AT) and Gastrocnemius Muscles (FIG. 4)

The weight of the AT (FIG. 4A) and gastrocnemius (FIG. 4B) muscles were evaluated in non-immobilised mice (control group), and after 14 days of immobilisation in mice treated either by the vehicle or by the compound BIO101 throughout the duration of immobilisation. As expected, it is observed that the immobilisation causes a reduction in the muscle mass of the AT (−21.7%, p<0.001) in mice that received the vehicle compared with a control group, non-immobilised (FIG. 4A).

It is observed that the weight of the AT and gastrocnemius muscles does not vary significantly in the test group of the mice treated with the compound BIO101 compared with the vehicle-treated reference group (FIGS. 4A and 4B).

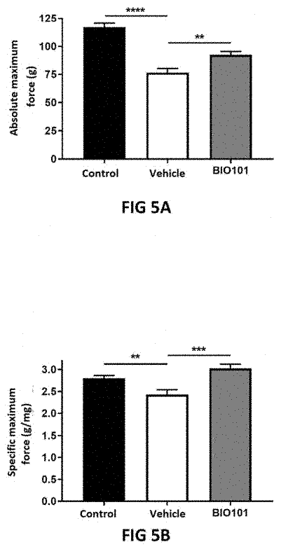

### Absolute and Specific Maximum Isometric Force of the Anterior Tibial Muscle (in situ Functional Study (FIGS. 5 and 6))

An evaluation of the contractility in situ of the AT muscle is made at various times in the protocol: on non-immobilised control mice (control group, J0), on mice subjected to immobilisation of the posterior paws for 7 days (J7), 14 days (J14), immobilised for 14 days and then remobilised for 2 weeks (J28).

When the force developed by the AT muscle is considered after seven days of immobilisation, as expected it is found that the animals treated with the vehicle (reference group) have an absolute maximum isometric contraction force significantly less than that of the non-immobilised control animals (−34.7%, p<0.001) (FIG. 5A). The animals treated with the compound BIO101 exhibit a loss of absolute force that is less (−21.1%, p=0.001) compared with the control, than the animals treated with the vehicle (FIG. 5A).

Interestingly, it is observed that the treatment with the compound BIO101 enables the animals immobilised for 7 days to keep a significantly greater absolute maximum isometric force and improves their performance (+21%, p=0.01) compared with animals treated with the vehicle, and this despite the absence of any effect of the compound BIO101 on the loss of mass.

It is observed that the animals treated with the vehicle have a specific maximum isometric contraction force (sP0; FIG. 5B) significantly less than that of the non-immobilised control animals (−13.2%, p<0.01). Remarkably, the specific maximum isometric force of the animals treated with the compound BIO101 is not significantly affected by 7 days of immobilisation: this is because the treatment with the compound BIO101 enables the animals immobilised for 7 days to keep a normal muscle function compared with the animals of the immobilised group, treated with the vehicle (+24.3%, p<0.001).

At the moment the immobilisation stops, at J14, the mice that receive the BIO101 treatment have lost only 22.4% (p<0.001) of the absolute maximum isometric force compared with the non-immobilised control mice (J0), as against 34% (p<0.001) for the mice that received the vehicle. Treatment with BIO101 limits the loss of absolute maximum isometric force (+17.5%, p<0.05) compared with mice treated with the vehicle (FIG. 6A).

Concerning the specific maximum isometric force, the mice that received the BIO101 treatment do not lose any force 14 days post immobilisation compared with the non-immobilised control mice (+5%, 2.94 g/mg versus 2.80 g/mg respectively, p=ns).

The mice treated with the vehicle for their part lose 11.3% of their specific force compared with the non-immobilised mice (p=0.06).

After 14 days of immobilisation, the treatment with BIO101 tends to limit the loss of specific maximum force (+18.4%, ns) compared with mice treated with the vehicle (FIG. 6B).

Because of the properties of phytoecdysones and derivatives thereof on the muscle function of mammals subjected to immobilisation, the use of phytoecdysones and derivatives thereof can therefore be proposed, for preserving muscle function, in particular with regard to muscle force, and thus slowing down the loss of muscle functions related to immobilisation.

What is claimed is:

1. A method for preventing loss of muscle strength during immobilization in mammals comprising administering to a mammal a pharmaceutically acceptable formulation suitable for oral administration including at least one phytoecdysone or at least one semisynthetic derivative of a phytoecdysone.

2. The method according to claim 1, wherein the formulation includes 20-hydroxyecdysone or a semisynthetic derivative of 20-hydroxyecdysone.

3. The method according to claim 1, wherein the formulation includes a compound of general formula (I): 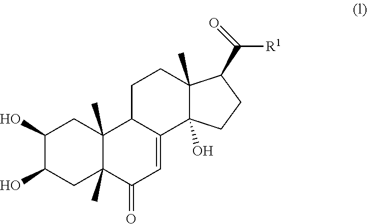
wherein:
R1 is chosen from: a (C1-C6)W(C1-C6) group; a (C1-C6)W(C1-C6)W(C1-C6) group; a (C1-C6)W(C1-C6)CO2(C1-C6) group; a (C1-C6)A group, A representing a heterocycle, optionally substituted by a group chosen from OH, OMe, (C1-C6), N(C1-C6), CO2(C1-C6); a CH2Br group;
W being a heteroatom chosen from N, O and S.

4. The method according to claim 1, wherein the formulation includes a compound chosen from the following compounds:
n° 1: (2S,3R,5R,10R,13R,14S,17S)-2,3,14-trihydroxy-10,13-dimethyl-17-(2-morpholinoacetyl)-2,3,4,5,9,11,12,15,16,17-decahydro-1H-cyclopenta[a]phenanthren-6-one,
n° 2: (2S,3R,5R,10R,13R,14S,17S)-2,3,14-trihydroxy-17-[2-(3-hydroxypyrrolidin-1-ypacetyl]-10,13- dimethyl-2,3,4,5,9,11,12,15,16,17-decahydro-1H-cyclopenta[a]phenanthren-6-one;
n° 3: (2S,3R,5R,10R,13R,14S,17S)-2,3,14-trihydroxy-1742-(4-hydroxy-1-piperidyl[acetyl]-10,13-dimethyl-2,3,4,5,9,11,12,15,16,17-decahydro-1H-cyclopenta[a]phenanthren-6-one;
n° 4: (2S,3R,5R,10R,13R,14S,17S)-2,3,14-trihydroxy-17-[2-[4-(2-hydroxyethyl)-1-piperidyl]acetyl]-10,13-dimethyl-2,3,4,5,9,11,12,15,16,17-decahydro-1H-cyclopenta[a]phenanthren-6-one;
n° 5: (2S,3R,5R,10R,13R,14S,17S)-17-[2-(3-dimethylaminopropyl(methyl)amino)acetyl]-2,3,14-trihydroxy-10,13-dimethyl-2,3,4,5,9,11,12,15,16,17-decahydro-1 H-cyclopenta[a]phenanthren-6-one;
n° 6: ethyl 2-[2-oxo-2-[(2S,3R,5R,10R,13R,14S,17S)-2,3,14-trihydroxy-10,13-dimethyl-6-oxo-2,3,4,5,9,11,12,15,16,17-decahydro-1H-cyclopenta[a]phenanthren-17-yl]ethyl]sulfanylacetate;
n° 7: (2S,3R,5R,10R,13R,14S,17S)-17-(2-ethylsulfanylacetyl)-2,3,14-trihydroxy10,13-dimethyl-2,3,4,5,9,11,12,15,16,17-decahydro-1 H-cyclopenta[a]phenanthren-6-one;
n° 8: (2S,3R,5R,10R,13R,14S,17S)-2,3,14-trihydroxy-17-[2-(2-hydroxyethyl sulfanyl)acetyl]-10,13-dimethyl-2,3,4,5,9,11,12,15,16,17-decahydro-1H cyclopenta[a]phenanthren-6-one.

5. The method according to claim 1, wherein the formulation includes a compound of formula (II):

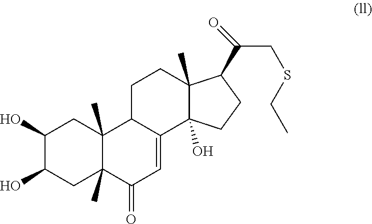 [SMILES: CCSCC(=O)[C@H]1CC[C@@]2(O)C3=CC(=O)[C@]4(C)C[C@@H](O)[C@@H](O)C[C@]4(C)C3CC[C@]12C]

6. The method according to claim 1, wherein the phytoecdysones are administered at a dose of between 50 and 1000 milligrams per day in humans.

7. The method according to claim 1, wherein the formulation is administered during immobilisation.

8. The method according to claim 1, wherein the formulation is administered until immobilisation ends.

9. The method according to claim 7, wherein the formulation is also administered during a predetermined period after ending of immobilisation.
